(19) United States
(12) Patent Application Publication
(10) Pub. No.: US 2010/0125262 A1
(43) Pub. Date: May 20, 2010

(54) Odor Control in Personal Care Products

(75) Inventors: John Gavin MacDonald, Decatur, GA (US); Roger Bradshaw Quincy, III, Cumming, GA (US)

Correspondence Address: DORITY & MANNING, P.A., POST OFFICE BOX 1449, GREENVILLE, SC, 29602-1449, US

(73) Assignee: KIMBERLY-CLARK WORLDWIDE, INC., Neenah, WI (US)

(21) Appl. No.: 12/691,102

(22) Filed: Jan. 21, 2010

Related U.S. Application Data
(62) Division of application No. 10/723,761, filed on Nov. 26, 2003.

(51) Int. Cl. A61L 15/46

(52) U.S. Cl. 604/359

(57) ABSTRACT

This invention concerns an odor control layer for personal care products having a dried, aqueously deposited formulation of odor sorbent and binder. This layer may be placed in a personal care product like diapers, training pants, absorbent underpants, adult incontinence products, and feminine hygiene products. The amount of odor sorbent present may be in an amount of between about 2 and 80 weight percent on a dry basis. The layer may be a tissue, film, paper towel, nonwoven web, coform, airlaid, wet-laid, bonded-carded web and laminates thereof.

### BACKGROUND OF THE INVENTION

The invention concerns processes and products for the alleviation and control of odors in personal care products.

Disposable personal care products perform a needed function in today's busy society, freeing caregivers users from the chore of washing reusable products and allowing for the quick and easy disposal of body wastes. As leakage issues have been reduced because of improved designs, the control of odors has become more important for the consumer. This is a particular concern to users of incontinence and feminine hygiene products.

Odor is often used by consumers as a signal that a personal care product should be changed. The detection of the odor depends, however, on the acuity of the sense of smell of the consumer, an acuity that often declines with age. Relying on the odor of the product also means that the odor must become offensive before the product is changed, an unacceptable signal.

It is also important that anything added to a personal care product to reduce odor should remain in place and not migrate through the product, as has occurred in previous attempts to address this matter. Absorbent and/or adsorbent (sorbent) particles, for example, should not escape from the product nor be noticeable to the consumer.

It is clear that there exists a need for a process and product which allows for the control of odors due to bodily wastes in personal care products.

### SUMMARY OF THE INVENTION

In response to the foregoing difficulties encountered by those of skill in the art, we have invented an odor control layer for personal care products having a dried, aqueously deposited formulation of odor sorbent and binder. This layer may be placed in a personal care product like diapers, training pants, absorbent underpants, adult incontinence products, and feminine hygiene products. The amount of odor sorbent present may be in an amount of between about 2 and 80 weight percent on a dry basis. The layer may be a tissue, film, paper towel, nonwoven web, coform, airlaid, wet-laid, bonded-carded web and laminates thereof.

The invention includes feminine hygiene products and adult incontinence products having a liquid impervious baffle, a liquid pervious body side liner, and a substrate having thereon a dried, aqueously applied layer of odor sorbent and binder.

A personal care product having the inventive durable odor sorbent treatment has odor reduction superior to a similar product lacking such an odor sorbent treatment. The pouch commonly used for disposal of a personal care product may also have an odor absorbing treatment. An odor reduction insert for air barrier packaging may also have a substrate with an odor reduction treatment of odor sorbent and binder.

The invention also encompasses a method of controlling odor in a personal care product having the steps of dipping a substrate into a formulation containing odor sorbent, binder and water, drying the substrate, and placing the substrate into a personal care product.

### BRIEF DESCRIPTION OF THE DRAWINGS

FIG. 1 is a drawing of a pouch containing a personal care product.

FIG. 2 is a drawing of a cross section of the pouch of FIG. 1

FIG. 3 is a drawing of a package of toilet paper.

FIG. 4 is a drawing of a feminine hygiene product.

FIG. 5 is a drawing of an adult incontinence product.

FIG. 6 is a drawing of a cross-section of an adult incontinence product.

FIG. 7 is a drawing of an absorbent underpant.

FIG. 8 is a diagram of a method of treating a fabric.

### DETAILED DESCRIPTION OF THE INVENTION

The present invention involves the control of odors in personal care products, i.e., diapers, training pants, absorbent underpants, adult incontinence products, and feminine hygiene products.

The control of odors in personal care products is of particular interest to adults like those who wear feminine hygiene pads and incontinence products. The desire to avoid embarrassment due to unpleasant odors is important to adult consumers of these products and the instant invention helps greatly in this regard.

The inventors have found a way to produce a durable treatment of an odor sorbent onto a layer in a personal care product. The odor sorbent is deposited onto the layer and dried from a formulation that includes the odor sorbent, binder and water. This odor absorbing formulation may be deposited using a number of methods and remains substantially in place despite the rigors of product use.

The odor sorbent may be zeolites, silicas, aluminas, titanias, sodium carbonates, sodium bicarbonates, sodium phosphates, zinc and copper sulfates and activated carbon in particle or fiber form, or other chemicals known to control odors, and mixtures thereof. The amount of odor sorbent will vary depending on the effectiveness of the absorbent chosen but should generally be in the range of about 2 to about 80 weight percent, desirably between about 5 and 75 weight percent and more desirably between about 10 and 30 weight percent.

Examples of formulations available that contain sorbents include Nuchar PMA Ink from MeadWestvaco Corporation of New York, N.Y., USA. Other sorbent products are available from the Calgon Carbon Corporation of Pittsburgh, Pa., USA, under the trade name CARBABSORB®, from Sigma-Aldrich Chemical Company of Milwaukee, Wis., USA and from Cabot Corporation of Boston, Mass., USA.

Personal care products in which the inventive odor sorbent substrate may be placed include feminine hygiene products, incontinence products and absorbent underpants.

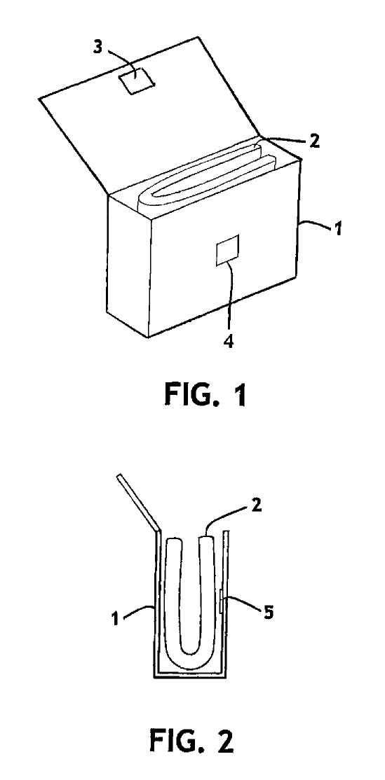

Personal care products, particularly feminine hygiene products, are often disposed of by placing them in the small pouch in which the product is commonly packaged for sale. The sorbent may also be placed within such a pouch to help reduce odor for disposal. FIG. 1 shows a pouch 1 within which is a personal care product 2. The pouch has fastening means 3, 4 such as conventional hook and loop fasteners or adhesive to resealably open the pouch. A cross section taken along the long dimension of the package (FIG. 2) shows the pouch 1 within which is a folded personal care product 2, and the odor is absorbent layer 5 within the pouch 1.

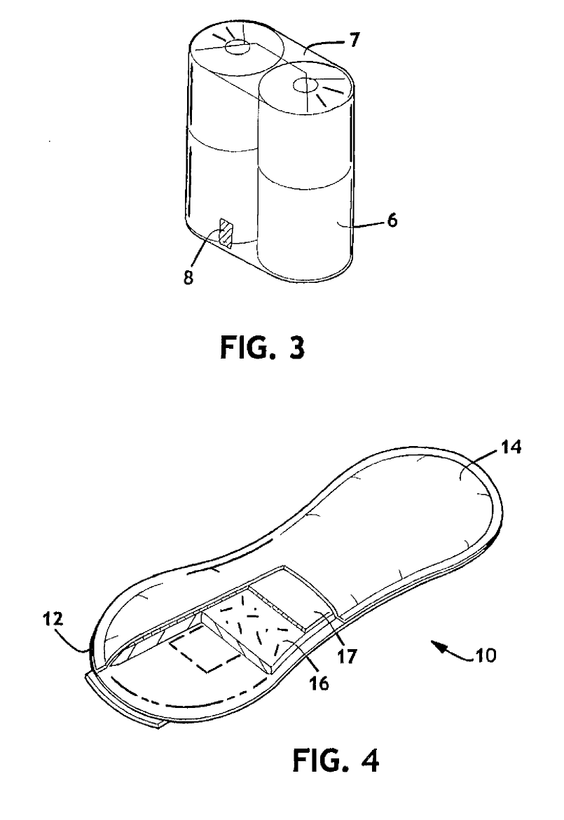

The odor sorbent substrate may also be placed within packaging for products where the packaging is an air or oxygen barrier, to absorb and/or adsorb odors from the components of the item being packaged. Such an odor reduction insert can help reduce the odors that build up within such packaging during storage and shipping of products like toilet paper and paper towels. For example, FIG. 3 shows a package of toilet paper rolls 6 in a plastic wrap 7. The odor reduction insert 8 is visible within the package.

The odor sorbent may be included in feminine hygiene products include as mentioned above. These include, for example, the pad shown partially cut away in FIG. 4. This pad 10 has a liquid impervious baffle 12 on the side away from the wearer. The baffle 12 is often made from a film like a polyethylene or polypropylene film. The layer closest to the wearer is the liner 14 and is a liquid permeable layer that is preferably soft and absorbent. Between the baffle 12 and liner 14 there may be a number of layers for different purposes, such as an absorbent core 16 designed to hold the majority of any liquid discharge. Other optional layers include a transfer delay layer 17, and tissue wraps (not shown).

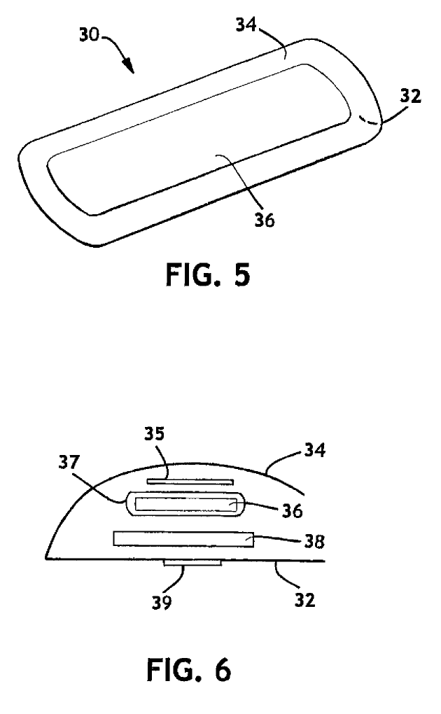

Incontinence products 30 as shown in FIG. 5 likewise have a baffle or outer cover 32, an innermost liner 34 and various layers in between, like the absorbent core 36. FIG. 6 shows an incontinence product in cross-section where the section is taken across the narrow part of the product. The liner 34 is at top, underlain by a surge layer 35 which acts like a reservoir to accept large surges of liquid and slowly release them to the subsequent layers. Below the surge layer 35 is an absorbent core or pledget 36 surrounded by tissue wrap 37. The absorbent core of some products contains superabsorbent particles which are loose and very small and which may escape onto the body or clothing unless contained. The tissue wrap 37 surrounds the core 36 and keeps the superabsorbent particles from leaving the core 36. Under the tissue wrapped core is a fluff layer 38 and then the baffle 32. Many products also have an adhesive strip 39 to help hold the product in place in use by adhering it to the user's underclothes. More information concerning incontinence products may be found, for example, in U.S. Pat. Nos. 4,940,464 and 4,938,753 which are incorporated herein in their entirety by reference thereto for all purposes.

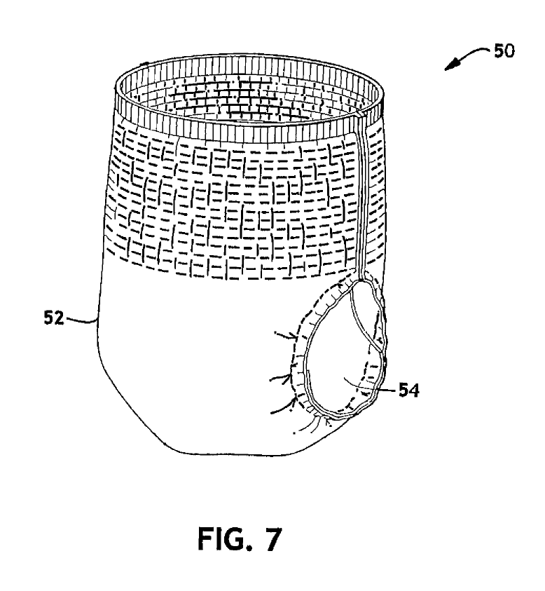

Absorbent underpants 50 as shown in FIG. 7 have a baffle 52, liner 54 and absorbent core (not shown), A further discussion regarding absorbent underpants may be found, for example, in U.S. Pat. Nos. 6,240,569 and 6,367,089 which are incorporated herein in their entirety by reference thereto for all purposes.

The odor sorbent of the invention may be applied onto a fabric layer from an aqueously based formulation, dried, and the dried layer placed in the product. Alternatively, the formulation containing the sorbent may be applied onto an existing layer within the product, like the absorbent core, and allowed to dry. Substrates suitable for treatment with the sorbents of the invention include films, tissues, paper towels, woven and nonwoven fabrics, coform materials, airlaid materials, wet-laid materials, bonded-carded webs and so forth. Nonexclusive examples of substrates may be found in U.S. Pat. Nos. 4,775,582 and 4,853,281, U.S. Pat. No. 4,833,003, and U.S. Pat. No. 4,511,488, all assigned to the Kimberly-Clark Corporation.

A nonwoven fabric may be made according to processes like spunbonding, meltblowing, airlaying, bonding and carding, and so forth. Nonwoven fabrics may be made from thermoplastic resins including, but not limited to polyesters, nylons, and polyolefins. Olefins include ethylene, propylene, butylenes, isoprene and so forth, as well as combinations thereof.

The term “coform” means a process in which at least one meltblown diehead is arranged near a chute through which other materials are added to the web while it is forming. Such other materials may be pulp, superabsorbent particles, natural polymers (for example, rayon or cotton fibers) and/or synthetic polymers (for example, polypropylene or polyester) fibers, for example, where the fibers may be of staple length. Coform processes are shown in commonly assigned U.S. Pat. No. 4,818,464 to Lau and U.S. Pat. No. 4,100,324 to Anderson et al. Webs produced by the coform process are generally referred to as coform materials.

A bonded carded web is made from staple fibers which are sent through a combing or carding unit, which breaks apart and aligns the staple fibers in the machine direction to form a generally machine direction-oriented fibrous nonwoven web. Once the web is formed, it then is bonded by one or more of several methods such as powder bonding, pattern bonding, through air bonding and ultrasonic bonding.

In the airlaying process, bundles of small fibers having typical lengths ranging from about 3 to about 52 millimeters (mm) are separated and entrained in an air supply and then deposited onto a forming screen, usually with the assistance of a vacuum supply. The randomly deposited fibers then are bonded to one another. Examples of airlaid teachings include the DanWeb process as described in U.S. Pat. No. 4,640,810 to Laursen et al. and assigned to Scan Web of North America Inc, the Kroyer process as described in U.S. Pat. No. 4,494,278 to Kroyer et al. and U.S. Pat. No. 5,527,171 to Soerensen assigned to Niro Separation a/s, the method of U.S. Pat. No. 4,375,448 to Appel et at assigned to Kimberly-Clark Corporation, or other similar methods.

The sorbent may be applied to the substrate layer by a fluid saturation method such as the dip and squeeze method, which entails dipping the layer into a formulation having the sorbent and binder, squeezing out the excess, and drying.

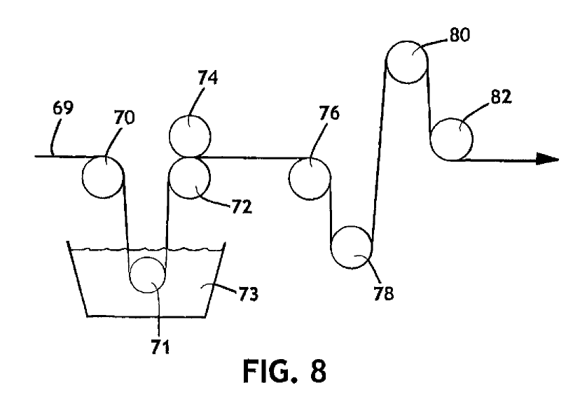

The sorbent may be applied to the layer with a saturation treater and then dried with, for example, steam cans. This method is illustrated in FIG. 8 wherein a tissue layer 69 travels around rollers 70, 71 through a reservoir 73 and then between a rubber applicator roll 72 and a stainless steel pick-up roll 74 where it is “nipped” or squeezed and excess liquid removed. The wet tissue layer 70 is then dried over four steam cans 76, 78, 80, 82. In one example, the nip pressure between the pick-up and applicator rolls was 92 psi (634 Kilopascals, KPa), the amount of odor sorbent and binder applied was in the range of 100 to 127 weight percent, the feed rate was 28 ft/min (8.53 m/min) and the steam can temperatures were, respectively, 176 F, 170 F, 185 F and 191 F (80.0, 76.7, 85.0, and 88.3 C). Alternatively the wetted tissue may be dried by other means such as through the use of through-air drying.

In order to test the effectiveness of applying the sorbent by coating versus saturation, both methods were performed and the resulting material tested. In this test, Nuchar PMA ink was applied to a wetlaid cellulosic fabric using different surface coating methods including using a blade and a Meyer rod (No. 10 double wound). Other samples of the same fabric were saturated as described above and dried with steam cans. The odor absorption efficiency was measured using the GC headspace method with pyridine (amine) as the model odor and was conducted on an Agilent 5890, Series II gas chromatograph with an Agilent 7694 headspace sampler, both available from Agilent Technologies, Waldbronn, Germany. Helium was used as the carrier gas (injection port pressure: 12.7 psig (188.9 kPa); headspace vial pressure: 15.8 psig (210.3 kPa); supply line pressure: 60 psig (515.1 kPa)), A DB-624 column that had a length of 30 m and an internal diameter of 0.25 mm was used for the odorous compound (available from J&W Scientific, Inc. of Folsom, Calif.).

The operating parameters used for the headspace gas chromatography are shown below in the table below.

### Operating Parameters for the Headspace Gas Chromatography Device

Headspace Parameters

| | | |
|---|---|---|
| Zone Temps, ° C. | Oven | 37 |
| | Loop | 42 |
| | TR. Line | 47 |
| Event Time, minutes | GC Cycle | 10.0 |
| | time | |
| | Vial eq. Time | 10.0 |
| | Pressuriz. | 0.20 |
| | Time | |
| | Loop fill time | 0.20 |
| | Loop eq. | 0.15 |
| | Time | |
| | Inject time | 0.30 |
| Vial Parameters | First vial | 1 |
| | Last vial | 1 |
| | Shake | [off] |

The test procedure involved placing 0.005-0.006 g of a sample containing the odor absorbing agent in a 20 cubic centimeter (cc) headspace vial. Using a syringe, an aliquot of the odorous compound was also placed in the vial. The vial was then sealed with a cap and a septum and placed in a headspace gas chromatography oven at 37° C. After ten minutes, a hollow needle was inserted through the septum and into the vial. A 1 cc sample of the headspace (air inside the vial) was then injected into the gas chromatograph.

The results of the testing are shown below and it should be noted that due to the mildly acidic nature of the cellulose in the wetlaid fabric, the control does absorb some of the pyridine.

| Sample | Appl. Method | % carbon | mg pyridine/g |
|---|---|---|---|
| Wetlaid control | NA | NA | 53 |
| Wetlaid/PMA carbon | one side blade | 3.3 | 54 |
| Wetlaid/PMA carbon | one-side rod | 6.3 | 60 |
| Wetlaid/PMA carbon | one side blade | 10.9 | 75 |
| Wetlaid/PMA carbon | dip & nip saturator | 10.0 | 90 |

The results show a sizeable increase in the absorption of pyridine by saturation of the substrate compared to surface coating.

The formulation of the invention containing sorbent and binder dries to produce a durable treatment that will not migrate or fall off when in use or transport. Durability may be measured by placing the substrate between the thumb and forefinger and rubbing the two together. Little or no sorbent should be left on the fingers. Another test, widely used in the flexographic printing industry, is to place the treated substrate on a hard surface, place one's thumb on the substrate, and rotate the thumb about 90 degrees. Again, little or no sorbent should be left on the thumb. This “thumb twist” test is further described in C Lowi, G. Webster, S. Kellse and I. McDonald's “Chemistry & Technology for UV & EB Formulation for Coatings, Inks & Paints” volume 4, p. 54, published in 1997 by John Wiley & Sons Ltd. in association with SITA Technology, Ltd., ISBN 0 947798 54 4, and in C. Lowe and R. K .T. Oldring's “Test Methods for UV and EB Curing Systems”, volume 6, published in 1998 by John Wiley and Sons Ltd in association with SITA Technology Ltd., ISBN 0471 978906. This test is subject to some variability as the pressure applied by a particular tester may vary, but is surprisingly accurate under most conditions. This test may be correlated generally with the Taber Abrasion test which measures the number of cycles required for an abrasion wheel to wear completely through a fabric.

In the Taber Abrasion test a sample of fabric is placed on a turntable that rotates in the horizontal plane while an abrasive wheel rests on the sample as it turns. The wheel turns at the same rate as the turntable which turns at a rate of about 30 to 45 revolutions per minute. Wheels of varying degrees of abrasiveness are available. The Taber Abrasion testing device is available from Teledyne Taber, North Tonawanda, N.Y., USA as model number 5130, with an H-38 wheel and 125 gram counterweight. In this configuration the samples according to the invention should endure at least 10 cycles without a visible amount of sorbent being transferred to the wheel.

The odor sorbent coated substrate may be placed within a personal care product in any number of locations. The substrate may, for example, be placed immediately below the liner, below the surge, between the core and fluff or below the fluff. The substrate may replace the tissue wrap or be a secondary wrap for the core.

The following examples aid in understanding the invention.

Experiment 1

Poise® Regular size pads from Kimberly-Clark Corporation of Dallas, Tex., USA were used in this study. Carbon ink DPX-7861-49A was supplied by MeadWestvaco and contained 15 weight percent carbon, 11 weight percent styrene-acrylic binder and 74 weight percent water.

### EXAMPLE 1

MeadWestvaco DPX-7861-49A ink was coated onto 11 cm by 16 cm strips of Hi-Count® 1-ply tissue. Each strip after drying had approximately 32 mg of the carbon/binder with about 18 mg being carbon. A strip was placed below the fluff layer and above the baffle in an adult incontinence article (see FIG. 6), in this example Poise® Pads, size regular, available from Kimberly-Clark Corporation of Dallas, Tex., USA.

### EXAMPLE 2

Activated carbon and binder ink (from MeadWestvaco under the designation DPX-7861-49A) was coated onto 3.5 cm by 26 cm strips of Hi-Count® 1-ply tissue and dried. Each strip after drying had approximately 6.6 mg of carbon. The strip was wrapped around the absorbent core pledget lengthwise, leaving the sides of the pledget open. This was done in an adult incontinence article (see FIG. 5), in this example Poise® Pads, size regular, available from Kimberly-Clark Corporation of Dallas, Tex., USA.

### EXAMPLE 3

Activated carbon and binder ink (from MeadWestvaco under the designation DPX-7861-49A) was coated onto 3.4 cm by 9 cm strips of a wet laid layer. Each strip after drying had about 26 mg of carbon. Strips were placed at both ends of the absorbent core of an adult incontinence article (see FIG. 5), in this example Poise® Pads, size regular, available from Kimberly-Clark Corporation of Dallas, Tex., USA.

### EXAMPLE 4

Activated carbon and binder ink (from MeadWestvaco under the designation DPX-7861-49A) was coated onto 11 cm by 14 cm pieces of the tissue wrap normally used in a Poise® pad to wrap the absorbent core. Each strip after drying had about 14 mg of carbon. The coated tissue wrap was used in the normal location for tissue wrap, i.e., wrapped around the absorbent core in an adult incontinence article (see FIG. 6), in this example Poise® Pads, size regular, available from Kimberly-Clark Corporation of Dallas.

Control 1

A commercially available Poise® Pad similar to that of the Examples but lacking the inventive carbon odor sorbent layer.

Control 2

A Serenity® Night & Day with Odasorb plus™, size extra plus, commercially available from Serac LLC of Eddystone, Pa., USA, a subsidiary of SCA Hygiene Products of Munich, Germany. These pads were cut into two substantially equal pieces so that the weight and size of the pads would approximate that of the regular size Poise® pads.

The Examples and the Controls described in Experiment 1 were then assessed for odor by a sensory panel according to ASTM E1207-87 (Standard Practice for the Sensory Evaluation of Axillary Deodorancy) yielding the results in Table 1 below. In the test, each sample was insulted with 60 ml of pooled female urine and incubated for 24 hours at 37° C. in a closed, 1 quart (0.95 liter), glass container. Twelve trained female panelists ranked the pads in order of urine odor intensity.

TABLE 1
order from least to most odor.

| Sample | Odor Ranking |
|---|---|
| Example 4 | 8.6 (least odor) |
| Example 3 | 9.3 |
| Example 2 | 16.4 |
| Control 1 | 16.6 |
| Example 1 | 17.2 |
| Control 2 | 31.8 (most odor) |

Experiment 2

In the following experiment, Poise® Extra Plus size pads were used in the study. The carbon ink used was Nuchar PMA having 15 weight percent carbon, 12 weight styrene-acrylic copolymer binder and 73 weight percent water and was supplied by MeadWestvaco.

### EXAMPLE 5

Activated carbon and binder ink (Nuchar PMA) was coated onto 6 cm by 22 cm pieces of a polyethylene film that were then placed into a Poise® pad just above the baffle, i.e., on the side toward the wearer. Each strip after drying had about 20 mg of carbon. The tissue wrapped pledget (untreated) was placed under the fluff baffle.

### EXAMPLE 6

Activated carbon and binder ink (Nuchar PMA) was coated onto 6 cm by 20.5 cm pieces of the tissue wrap normally used in a Poise® pad to wrap the absorbent core.

Each strip after drying had about 20 mg of carbon. The coated tissue strip was placed just below the surge layer in a Poise® pad, regular size. The tissue wrapped pledget (not treated) was placed under the fluff baffle.

### EXAMPLE 7

Activated carbon and binder ink (Nuchar PMA) was coated onto a 13 cm by 20.5 cm pieces of tissue wrap normally used in a Poise® pad to wrap the absorbent core. Each strip after drying had about 40 mg of carbon. The pledget was wrapped with the treated tissue and then placed in the Poise® pad under the baffle on the garment side.

Control 3

Commercially available Poise® extra plus size pads were used without any changes. Note these pads have the tissue wrapped pledget just under the surge layer.

Control 4

Poise® extra plus size was prepared having the tissue wrapped pledget inserted under the fluff baffle.

Control 5

Serenity® Night & Day with Odasorb plus™, extra plus size, was used unchanged.

The Examples and Controls described in Experiment 2 were then assessed for odor by a sensory panel according to ASTM E1207-87 yielding the results in Table 2. In the test, each sample was insulted with 60 ml of pooled female urine and incubated for 24 hours at 37 C in a closed 1 quart (0.95 liter) glass container. Twelve trained female panelists ranked the pads in order of urine odor intensity.

TABLE 2
order from least to most odor.

| Sample | Odor Ranking |
|---|---|
| Example 6 | 7.4 (least odor) |
| Example 5 | 8.6 |
| Example 7 | 12.6 |
| Control 5 | 15.7 |
| Control 4 | 24.7 |
| Control 3 | 31.1 (most odor) |

Experiment 3

Poise® regular size pads were used for this study. Carbon ink from MeadWestvaco was used in this study.

### EXAMPLE 8

Activated carbon and binder ink (Nuchar PMA)) was coated onto 3 cm by 11 cm pieces of a polyethylene film that was then inserted into a Poise® pad just above the baffle, i.e., on the side toward a wearer. Each strip after drying had about 10 mg of carbon. The tissue wrapped pledget (untreated) was placed under the blue surge layer.

### EXAMPLE 9

Activated carbon and binder ink (DPX-7861-49A) was coated onto 11 cm by 14 cm pieces of the tissue wrap normally used in a Poise® pad to wrap the absorbent core. Each strip after drying had about 18 mg of carbon. The coated tissue wrap was used in the normal location for tissue wrap, i.e., wrapped around the absorbent core in an adult incontinence article (see FIG. 6)

### EXAMPLE 10

Activated carbon and binder ink (DPX-7861-49A) was coated onto 11 cm by 14 cm pieces of the tissue wrap normally used in a Poise® pad to wrap the absorbent core. Each strip after drying had about 7 mg of carbon. The coated tissue wrap was used in the normal location for tissue wrap, i.e., wrapped around the absorbent core in an adult incontinence article (see FIG. 6).

Control 6

A Serenity® Night & Day with Odasorb plus™, size extra plus, commercially available from Serac LLC of Eddystone, Pa., USA, a subsidiary of SCA Hygiene Products of Munich, Germany. These pads were cut into two so that the weight (and hence size) of the pads were similar to that of the regular Poise® pads.

Control 7

A commercially available Poise® Pad similar to that of the Examples but lacking the inventive carbon odor sorbent layer.

The Examples and the Controls described above were then assessed for odor by a sensory panel according to ASTM E1207-87 (Standard Practice for the Sensory Evaluation of Axillary Deodorancy) yielding the results in Table 3 below. In the test, each sample was insulted with 60 ml of pooled female urine and incubated for 24 hours at 37° C. in a closed, 1 quart (0.95 liter), glass container. Twelve female panelists ranked the pads in order of urine odor intensity.

TABLE 3
order from least to most odor.

| Sample | Odor Ranking |
|---|---|
| Example 9 | 5.3 (Least odor) |
| Example 10 | 8.0 |
| Example 8 | 9.7 |
| Control 6 | 28.8 |
| Control 7 | 30.6 (Most odor) |

The data shows that the inventive odor sorbent layer successfully reduces the perceived odor of the product. Clearly the personal care product having the layer with the durable odor sorbent formulation had odor control superior to a similar personal care product lacking the durable odor sorbent, according to ASTM E1207-87. By the term “similar product” what is meant is a product which uses essentially the same manufacturing processes and materials as the inventive product but in which the inventive item is lacking. According to Webster's New Collegiate Dictionary (1980), “similar” means 1) having characteristics in common; strictly comparable, 2) alike in substance or essentials; corresponding. Using this commonly accepted meaning of the word similar, this term means that all other conditions are essentially the same, within manufacturing tolerances, except for the inventive conditions mentioned.

All of the substrates to which the formulation containing activated carbon and binder were applied had durable treatments according to the flexographic industries' thumb twist pressure test or the Taber Abrasion test. The sorbent is not durably attached to the substrates unless it was applied using a formulation containing a sorbent and binder.

As will be appreciated by those skilled in the art, changes and variations to the invention are considered to be within the ability of those skilled in the art. Examples of such changes are contained in the patents identified above, each of which is incorporated herein by reference in its entirety to the extent it is consistent with this specification. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

1-16. (canceled)

17. A feminine hygiene product comprising:
a liquid impervious baffle;
a liquid pervious liner;
an absorbent core positioned between the baffle and the liner; and
an odor sorbent substrate that is positioned adjacent to the liquid pervious liner, wherein the odor sorbent substrate is coated with a durable activated carbon ink that comprises activated carbon particles and at least one styrene binder.

18. The feminine care product of claim 17, wherein the activated carbon particles are present in an amount of between about 2 and 80 wt. % of the substrate on a dry basis.

19. The feminine care product of claim 17, wherein the activated carbon particles are present in an amount of between about 5 and 75 wt. % of the substrate on a dry basis.

20. The feminine care product of claim 17, wherein the activated carbon particles are present in an amount of between about 10 and 30 wt. % of the substrate on a dry basis.

21. The feminine care product of claim 17, wherein the substrate contains a nonwoven web.

22. The feminine care product of claim 17, wherein the substrate contains a wetlaid or airlaid paper web.

23. The feminine care product of claim 17, wherein the styrene binder is a styrene-acrylic binder.

24. The feminine care product of claim 17, wherein the ink consists essentially of the activated carbon particles and the binder.

25. The feminine care product of claim 17, wherein the ink is applied to the substrate as an aqueous solution.

26. The feminine care product of claim 17, wherein the activated carbon particles are present in the ink in a greater amount than the binder.

27. A feminine hygiene product comprising:
a liquid impervious baffle;
a liquid pervious liner that is coated with a durable activated carbon ink that comprises activated carbon particles and at least one styrene binder; and
an absorbent core positioned between the baffle and the liner.

28. The feminine care product of claim 27, wherein the activated carbon particles are present in an amount of between about 2 and 80 wt. % of the substrate on a dry basis.

29. The feminine care product of claim 27, wherein the activated carbon particles are present in an amount of between about 5 and 75 wt. % of the substrate on a dry basis.

30. The feminine care product of claim 27, wherein the activated carbon particles are present in an amount of between about 10 and 30 wt. % of the substrate on a dry basis.

31. The feminine care product of claim 27, wherein the substrate contains a nonwoven web.

32. The feminine care product of claim 27, wherein the substrate contains a wetlaid or airlaid paper web.

33. The feminine care product of claim 34, wherein the styrene binder is a styrene-acrylic binder.

34. The feminine care product of claim 27, wherein the ink consists essentially of the activated carbon particles and the binder.

35. The feminine care product of claim 27, wherein the ink is applied to the substrate as an aqueous solution.

36. The feminine care product of claim 27, wherein the activated carbon particles are present in the ink in a greater amount than the binder.
